(19) United States
(12) Patent Application Publication
(10) Pub. No.: US 2008/0132744 A1
(43) Pub. Date: Jun. 5, 2008

(54) Rhenium Catalyst on a Silanized Alumina Carrier and its Use in the Metathesis Reaction of Olefins

(75) Inventors: Cecilia Querci, Novara (IT); Aldo Bosetti, Vercelli (IT); Rinaldo Guerrini, Novara (IT); Francesco Panella, Pertengo-Vercelli (IT); Matteo Russo, Cerano-Novara (IT)

Correspondence Address: OBLON, SPIVAK, MCCLELLAND MAIER & NEUSTADT, P.C., 1940 DUKE STREET, ALEXANDRIA, VA, 22314, US

(73) Assignee: POLIMERI EUROPA S.p.A., Brindisi (IT)

(21) Appl. No.: 10/580,834

(22) PCT Filed: Oct. 26, 2004
(86) PCT No.: PCT/EP04/12098

§ 371 Date: Dec. 19, 2006

(30) Foreign Application Priority Data
Nov. 27, 2003 (IT) MI2003 A 002321

(51) Int. Cl. B01J 23/36; C07C 6/04

(52) U.S. Cl. 585/470; 502/355; 585/646

(57) ABSTRACT

A process is described for the preparation of a heterogeneous catalyst containing rhecnium as active component and C)an inert carrier medium. characterized in that said inert carrier is previously treated with a silanizing compound containing chlorine, before the laying of the active component on the carrier, and the activation of the heterogeneous catalyst takes place by means of thermal treatment followed by a rapid final cooling. The catalyst is particularly active in metathesis reactions of olefins.

The present invention relates to a process for the preparation of a heterogeneous catalyst containing rhenium as active component and alumina as inert carrier, characterized in that said inert carrier is previously subjected to silanization treatment with a compound containing chlorine, before the laying of the active component on the carrier, and the activation of the heterogeneous catalyst takes place by means of thermal treatment followed by a rapid final cooling.

The present invention also relates to the use of said catalyst in the metathesis reaction of olefins.

Metathesis reactions, also known as dismutation and disproportioning of olefins, are reactions of great practical interest which can be used, for example, for rebalancing the weight of olefins deriving from steam cracking.

When olefins are treated in the presence of suitable catalysts, they are converted to other olefins in a reaction inn which the (R¹R²C=) alkylidene groups are interchanged changed with a process with is schematically represented by the following equation:

$$\begin{matrix}{R}^{1} {R}^{2} C = {CR}^{1} {R}^{2} & & {R}^{1} {R}^{2} C = {CR}^{3} {R}^{4} \\ + & < -- -- -- -> & + \\ {R}^{3} {R}^{4} C = {CR}^{3} {R}^{4} & & {R}^{1} {R}^{2} C = {CR}^{3} {R}^{4}\end{matrix}$$

Heterogeneous catalysts essentially consisting of rhenium derivatives carried on inert materials (for example silica or alumina) are known to be active in the metathesis of olefins. For example, U.S. Pat. Nos. 3,641,189 and 3,676,520 describe the preparation of these materials and their use in the metathesis of olefins.

In the preparation of this catalyst, the active component is usually laid on the surface of the carrier medium by means of impregnation. In this reaction, the carrier is mixed with a solution in which the active component has been dissolved. When the solvent is removed by evaporation, the active component remains inside the particles of the carrier.

With these catalysts, however, it is necessary for the active component to be present in quantities ranging from 5 to 7% and, in spite of this, not particularly high conversions have been observed, and also, in the case of higher olefins, low selectivities which can often be attributed to secondary isomerization reactions of the double bonds (J. Mol. Cat: 46, 1988, 119-130 and App. Catal., 70, 1991, 295-3 06) 3 06).

It has now been found that it is possible to overcome the above disadvantages and obtain optimum catalyst performances, using much lower quantities of active component, by means of the catalyst of the present invention containing rhenium and an inert carrier such as alumina, characterized in that said carrier is subjected to a prior silanization treatment with a compound containing chlorine, before impregnation with the active component, and the activation of the heterogeneous catalyst takes place by means of thermal treatment followed by a rapid final cooling.

This catalyst is active in metathesis reactions even when used without a co-catalyst and reduces problems relating to the formation of isomers or secondary reactions obtaining a high selectivity.

In accordance with what is specified above, an object of the present invention therefore relates to a process for the preparation of a heterogeneous catalyst active in metathesis reactions of olefins containing rhenium as active component and alumina as inert carrier medium, characterized in that the inert carrier medium is treated with a silanizing agent having the general formula RnSiClm (I) wherein R represents an amine or a Cl-C₂₅ (iso)alkyl, C₅-c₂₅ cyclo-alkyl, C6-C18 aromatic or C7-C25 alkyl aromatic radical, optionally containing at least one heteroatom selected from O, S and N; n is an integer so that 1 <n <3; m is an integer so that 1<m <3.

Examples of silanizing agents having general formula (I) are: trimethyl chloro silane, allyl trichloro silane, triphenyl chloro silane, tributyl chloro silane.

The treatment of the carrier is carried out using the silanizing agent as such or, preferably, by dissolution of the silanizing agent itself in a solvent selected from an alkyl or aromatic hydrocarbon such as hexane, heptane, octane, decane, toluene or xylenes; an alkyl or cyclic ether such as ethyl ether, dimethyl ether or tetrahydrofuran; a chlorinated product such as methylene chloride, carbon tetrachloride or chloroform.

The alumina is maintained in the presence of the solution of silanizing agent for a time ranging from 0.5 to 24 hours, preferably from 8 to 15 hours, at a temperature ranging from −10 to 100° C. At the end of the impregnation, after evaporation of the solvent, the alumina can be optionally subjected to thermal treatment ranging from 400 to 600° C.

According to the present invention, the alumina is preferably used with a surface area >50 m²/g, preferably from 100 to 200 m²/g, and with a total cumulative pore volume greater than 0.1 ml/g, preferably from 0.3 to 0.8 ml/g.

The rhenium compound can be laid on the carrier pre-treated as described above, by means of precipitation or impregnation starting from precursors consisting for example of solutions of its salts or soluble complexes.

The precursors of the rhenium compound are selected from rhenium heptoxide, ammonium perrenate, tetra-alkyl ammonium perrenate, perrenic acid or from other compounds known to experts in the art.

Impregnation of the inert carrier is generally preferred, using a saturated solution of the rhenium compound, in a solvent selected from water or an organic solvent, for example a hydrocarbon, an alcohol or an ether.

The impregnation is preferably effected at a temperature ranging from 20 to 70° C. in order to increase the solubility of the rhenium salt; in this case the carrier is also heated to the same temperature.

After impregnation of the carrier with the metal, the catalyst is activated with a pre-calcination at a temperature ranging from 100 to 200° C. in a stream of dry air and a subsequent calcination at a temperature ranging from 300 to 6000C, first in a stream of dry air and then nitrogen. The cooling is effected in a stream of nitrogen over a period of time ranging from 5 to 30 minutes, preferably from 10 to 20 minutes.

In these catalysts, the rhenium is normally present in a quantity ranging from 1 to 20% by weight, preferably from 3 to 10% by weight with respect to the carrier.

In order to obtain a further improvement of the catalyst, it is possible, after the treatment described above, to wet it with a quantity of water equal to the porosity of the carrier and calcine it again according to the method described above.

The catalysts of the present invention can be used in metathesis reactions of olefins.

These reactions can be homo-metathesis (two equal olefins) or co-metathesis (two different olefins).

Olefins which can be subjected to the metathesis reaction a re mono-olefins with from 2 to 30 carbon atoms such as, for example, ethylene, propylene, butene, pentene, hexene; cyclo-olefins with from 5 to 20 carbon atoms, for example cyclo-pentene, cyclo-octene, norbornene; olefins with two or more unsaturations containing from 4 to 30 carbon atoms, for example 1,4-hexadiene, 1,7-octadiene, cyclo-olefins containing two or more unsaturations containing from 6 to 30 carbon atoms, for example 1,5-cyclo-octadiene, norbordiene, dicyclopentadiene.

Other olefins can be mono-olefins or olefins containing several unsaturations, linear or cyclic, carrying functional groups such as, for example, halogens or ester groups such as methyl oleate.

The metathesis reaction can be carried out either batchwise or in continuous by feeding the materials into a fluid bed or fixed bed reactor. The reaction conditions such as temperature, pressure and streams are selected in relation to the material fed and the end-product desired.

The metathesis reaction is generally carried out at a temperature ranging from 0 to 100° C., preferably from 25 to 60° C., and at a pressure of up to 10 MPa, preferably from 0.1 to 6 MPa, and can be carried out in gas phase or in liquid phase, with or without an organic solvent.

When a solvent is used, this is selected from ethers, aliphatic and aromatic hydrocarbons. Examples of these solvents are ethyl ether, hexane, heptane, toluene, etc.

The catalyst is normally dispersed in the reaction medium at a concentration ranging from 1 to 50% by weight, preferably from 1 to 10% by weight with respect to the reaction mixture.

The metathesis reaction can be optionally carried out in the presence of co-catalysts selected from metal alkyls such as, for example, tin tetra-alkyls (tin tetramethyl, tin tetra-ethyl, tin tetrabutyl) or other metal alkyls such as lead tetramethyl, lead tetra-ethyl, aluminum triethyl, chloro aluminum diethyl, as described in patent U.S. Pat. No. 3,855,338.

The following examples are illustrative but non-limiting limiting of the invention described.

### EXAMPLE 1

### Preparation of Catalyst A

10 g of y-alumina with a surface of 180 m²/g and a porosity of 0.5 ml/g, are pre-calcined in a muffle at 110° C. for 1 hour in a stream of air and subsequently at 550° C. for 4 hours in a stream of air.

The carrier is then wet with 5 ml of a hexane solution containing 0.087 g of SiMe₃Cl and is maintained at 25° C for 18 hours. The hexane is then evaporated maintaining the sample in an oven at 60° C. for 2 hours.

The carrier thus treated is calcined first at 110° C for 1 hour in a stream of dry air and then at 550° C for 3 hours in a stream of dry air and for 1 hour in a stream of nitrogen. After cooling for 15 minutes in a stream of nitrogen, the carrier is then wet four times with 5 ml of an aqueous solution containing 0.126 g of NH₄ReO₄, the water is evaporated, between one impregnation and the next, by keeping the sample in an oven at 60° C.

The catalyst thus prepared has a rhenium content of 3.5% by weight.

### EXAMPLE 2

### Use of Catalyst A in Metathesis.

358 mg of catalyst A prepared as described in example 1 and 12 ml of a solution consisting of 10 Al of co catalyst SnMe₄ in 100 ml of hexane, are charged, in an argon atmosphere, into a 150 ml tailed flask.

The resulting mixture is maintained under light stirring, at 25° C. for 10 minutes and 13 ml of 1-hexene are then added.

After 10 minutes, the reaction mixture is analyzed via gas chromatography using an internal standard. The following results are obtained:

conversion of 1-hexene 55% - selectivity of 5-decene 100%

### EXAMPLE 3 (Comparative)

### Preparation of Catalyst B

10 g of y-alumina with a surface of 180 m²/g and a porosity of 0.5 ml/g, are pre-calcined in a muffle at 110° C. for 1 hour in a stream of air and subsequently at 550° C. for 4 hours in a stream of air.

The carrier is then wet four times with 5 ml of an aqueous solution containing 0.28 g of NH₄ReO₄, the water is evaporated, between one impregnation and the next, by keeping the sample in an oven at 60° C.

The carrier is calcined first at 110° C. for 1 hour in a stream of dry air and then at 550° C. for 3 hours in a stream of dry air and for 1 hour in a stream of nitrogen. The reactor is then extracted from the muffle and cooled for 15 minutes in a stream of nitrogen

The catalyst thus prepared has a rhenium content of 7.5% by weight.

### EXAMPLE 4 (Comparative)

### Use of Catalyst B in Metathesis

358 mg of catalyst B prepared as described in example 3 and 23 ml of a solution consisting of 10 μl of cocatalyst SnMe₄ in 100 ml of hexane, are charged, in an argon atmosphere, into a 150 ml tailed flask.

The resulting mixture is maintained under light stirring, at 25° C. for 10 minutes and 26 ml of 1-hexene are then added.

After 30 minutes, the reaction mixture is analyzed via gas chromatography using an internal standard. The follow- ing results are obtained: - conversion of 1-hexene 70% selectivity of 5-decene 95%

### EXAMPLE 5(Comparative)

### Preparation of Catalyst C 10 g of y-alumina with a surface of 180 m2 /g and a porosity of 0.5 ml/g, are pre-calcined in a muffle at 110C for 1 hour in a stream of air and subsequently at 550° C. for 4 hours in a stream of air.

The carrier is then wet four times with 5 ml of an aqueous solution containing 0.126 g of NH₄ReO₄, the water is evaporated, between one impregnation and the next, by keeping the sample in an oven at 60° C.

The carrier is calcined first at 110° C. for 1 hour in a stream of dry air and then at 550° C. for 3 hours in a stream of dry air and for 1 hour in a stream of nitrogen. The reactor is then extracted from the muffle and cooled for 15 minutes in a stream of nitrogen The catalyst thus prepared has a rhenium content of 3.5% by weight.

### EXAMPLE 6 (Comparative)

### Use of Catalyst C in Metathesis

358 mg of catalyst C prepared as described in example 5 and 23 ml of a solution consisting of 10 μl of co- catalyst SnMe₄ in 100 ml of hexane, are charged, in an ar- gon atmosphere, into a 150 ml tailed flask.

The resulting mixture is maintained under light stirring, at 25° C. for 10 minutes and 26 ml of 1-hexene are then added.

After 10 minutes, the reaction mixture is analyzed via gas chromatography using an internal standard. The following results are obtained:

conversion of 1-hexene 15% selectivity of 5-decene 85%

### EXAMPLE 7 (Compatative)

### Preparation of Catalyst D

10 g of y-alumina with a surface of 180 m²/g and a po- rosity of 0.5 ml/g, are pre-calcined in a muffle at 110° C for 1 hour in a stream of air and subsequently at 550° C for 4 hours in a stream of air.

The carrier is then wet four times with 5 ml of an aqueous solution containing 0.260 g of Me₃SiOSiMe₃, and is maintained at 25° C. for 18 hours. The hexane is then evapo- rated maintaining the sample in an oven at 60° C. for 2 hours.

The carrier medium thus treated is calcined first at 110° C for 1 hour in a stream of dry air and then at 550° C for 3 hours in a stream of dry air and for 1 hour in a stream of nitrogen. After cooling for 15 minutes in a stream of nitrogen, the carrier is then wet four times with 5 ml of an aqueous solution containing 0.126 g of NH₄ReO₄, the water is evaporated, between one impregnation and the next, by keeping the sample in an oven at 600C.

The catalyst thus prepared has a rhenium content of 3.5% by weight. EXAMPLE 8 (Comparative) Use of catalyst D in metathesis 358 mg of catalyst D prepared as described in example 7 and 23 ml of a solution consisting of 10 Ail of co- catalyst SnMe₄ in 100 ml of hexane, are charged, in an ar- gon atmosphere, into a 150 ml tailed flask.

The resulting mixture is maintained under light stir- WO 2005/051534 PCT/EP2004/012098 ring, at 25° C for 10 minutes and 26 ml of 1-hexene are then added.

After 10 minutes, the reaction mixture is analyzed via gas chromatography using an internal standard. The follow- ing results are obtained: - conversion of 1-hexene 35% selectivity of 5-decene 98%

1. A process for the preparation of a heterogeneous catalyst active in metathesis reactions of olefins containing rhenium as active component and alumina as inert carrier medium, characterized in that comprising treating the inert carrier is treated with a silanizing agent having the general formula RnSiClm (I) wherein R represents an amine or a CI-C₂₅ (iso) alkyl, C₅-C₂₅ cyclo-alkyl, C₆-C₁₈ aromatic or C₇-C₂₅ alkyl aromatic radical, optionally containing at least one heteroatom selected from 0, S and N; n is an integer so that 1 <n <3; m is an integer so that 1 <m <3.

2. The process according to claim 1, wherein the treatment of the carrier is effected using the silanizing agent as such or by means of dissolution of the silanizing agent in a solvent, the alumina being maintained in the presence of the solution of the silanizing agent, for a time ranging from 0.5 to 24 hours, at a temperature ranging from -10 to 100° C., and subjecting the alumina to optional thermal treatment ranging from 400 to 600° C.

3. The process according to claim 1 or 2, wherein the alumina has a surface area greater than 50 m²/g and a total cumulative pore volume greater than 0.01 ml/g.

4. The process according to claim 3, wherein the alumina has a surface area ranging from 100 to 200 m²/g and a total cumulative pore volume ranging from 0.3 to 0.8 ml/g.

5. The process according to claim 1, wherein the active rhenium component is laid on the carrier pretreated as specified in claims 1 4, by means of precipitation or impregnation starting from its precursors in the form of solutions of its salts or soluble complexes.

6. The process according to claim 5, wherein the rhenium precursors are selected from rhenium heptoxide, ammonium perrenate, tetra-alkyl ammonium perrenate and perrenic acid.

7. The process according to claim 1, wherein the catalyst contains a quantity of rhenium ranging from 1 to 20% by weight with respect to the carrier.

8. The process according to claim 7, wherein the catalyst contains a quantity of rhenium ranging from 3 to 10% by weight.

9. The process according to claim 1, wherein the catalyst containing rhenium on a carrier medium, is activated with a pre-calcination at a temperature ranging from 100 to 200° C. in a stream of dry air and a subsequent calcination at a temperature ranging from 300 to 600° C. first in a stream of dry air and then nitrogen.

10. A process for the conversion of olefins by means of a metathesis reaction characterized in that it is carried out comprising converting olefins in the presence of a heterogeneous catalyst active in metathesis reactions of olefins containing rhenium as active component and alumina as inert carrier medium, characterized in that wherein the inert carrier is treated with a silanizing agent having the general formula RnSiCIm (I) wherein R represents an amine or a CI-C₂₅ (iso)alkyl, C₅-C₂₅ cyclo-alkyl, C₆-C₁8 aromatic or C₇-C₂₅ alkyl aromatic radical, optionally containing at least one heteroatom selected from 0, S and N; n is an integer so that 1 <n <3; m is an integer so that 1 <m <3.

11. The process according to claim 10, wherein the metathesis reaction can be is homo-metathesis or co-metathesis.

12. The process according to claim 10, wherein the olefins are selected from mono-olefins having from 2 to 30 carbon atoms, cyclo-olefins having from 3 to 20 carbon atoms, polyolefins having from 6 to 30 carbon atoms, and cyclo- polyolefins having from 5 to 30 carbon atoms.

13. The process according to claim 12, wherein the mono-olefins are selected from ethylene, propylene, butene, pentene, and hexene.

14. The process according to claim 12, wherein the cycloolefins are selected from cyclo-pentene, cyclo-octene, and norbomene.

15. The process according to claim 12, wherein the polyolefins are selected from 1,4-hexadiene and 1,7-octadiene.

16. The process according to claim 12, wherein the cyclopolyolefins are selected from 1,5-cyclooctadiene, norbordiene and dicyclopentadiene.

17. The process according to claim 12, wherein the monoolefins or polyolefins, linear or cyclic, can carry functional groups such as[[,]]f example, halogens or ester groups such as methyl oleate.

18. The process according to claim 10, wherein the metathesis reaction is carried out at a temperature ranging from 0 to 100 ° C and a pressure ranging from 0 to 10 MPa (0 to 100 bar).

19. The process according to claim 18, wherein the metathesis reaction is carried out at a temperature ranging from 25 to 60 ° C and a pressure ranging from 0.1 to 6 MPa (1 to 60 bar).

20. The process according to claim 10, wherein the metathesis reaction is carried out in gas phase or in liquid phase with or without a solvent selected from ethers, aliphatic and aromatic hydrocarbons.

21. The process according to claim 20, wherein the solvent is selected from ethyl ether, hexane, heptane, and toluene.

22. The process according to claim 10, wherein the quantity of catalyst ranges from 1 to5O% by weight with respect to the reaction mixture.

23. The process according to claim 22, wherein the quantity of catalyst ranges from 1 to I0% by weight with respect to the reaction mixture.

24. The process according to claim 10, wherein the metathesis reaction is carried out batchwise or in a continuous manner.
